(19) United States
(12) Patent Application Publication
(10) Pub. No.: US 2026/0166106 A1
(43) Pub. Date: Jun. 18, 2026

(54) Pharmaceutical compositions and methods for treatment of diabetes mellitus

(71) Applicants: Greg Macpherson, Auckland (NZ); Alexander Tups, Dunedin (NZ)

(72) Inventors: Greg Macpherson, Auckland (NZ); Alexander Tups, Dunedin (NZ)

(21) Appl. No.: 18/984,486

(22) Filed: Dec. 17, 2024

(30) Foreign Application Priority Data
Dec. 17, 2024 (NZ) 817294

(51) Int. Cl. A61K 36/605; A61K 31/7048; A61K 36/28; A61P 3/10

(52) U.S. Cl. CPC A61K 36/605; A61K 31/7048; A61K 36/28; A61P 3/10

(57) ABSTRACT

Disclosed herein are compositions and methods for the treatment or prevention of diabetes mellitus and/or one or more complications thereof.

### FIELD

The disclosure relates to compositions and methods for the treatment or prevention of diabetes mellitus and/or one or more complications thereof.

### BACKGROUND

Diabetes mellitus, also known as diabetes, includes a group of common endocrine diseases characterized by elevated blood glucose levels. In 2021, 537 million adults were living with diabetes. This number is projected to increase to 643 million by 2030 and 783 million by 2045. In 2021, diabetes was responsible for $966 billion in global health expenditure.

Diabetes creates a high risk of long-term complications related to damage of blood vessels, including but not limited to diabetic retinopathy, diabetic nephropathy and diabetic skin ulceration.

There is a clear need to provide compositions and methods for the treatment or prevention of diabetes and/or one or more complications thereof.

It is an object of the present disclosure to provide compositions and methods for the treatment or prevention of diabetes and/or one or more f complications thereof. It is an alternative object of the present disclosure to provide the public with a useful choice.

### SUMMARY

In one example there is provided a composition comprising:
a. dahlia extract
b. eriocitrin
c. mulberry extract.

In another example, there is provided a method of treating or preventing diabetes and/or one or more complications thereof comprising administering to a subject in need thereof a composition comprising:
a. dahlia extract
b. eriocitrin
c. mulberry extract.

In another example, there is provided the use of a composition comprising
a. dahlia extract
b. eriocitrin
c. mulberry extract
   in the manufacture of a medicament for the treatment or prevention of diabetes and/or one or more complications thereof.

In another example, there is provided a composition for use in the treatment or prevention of diabetes and/or one or more complications thereof comprising:
a. dahlia extract
b. eriocitrin
c. mulberry extract.

In one aspect the dahlia extract is a dahlia flower extract.

In one aspect the dahlia flower extract is Dahlia4®.

In one aspect the mulberry extract is a mulberry leaf extract.

In one aspect the eriocitrin is from Eriomin™.

### BRIEF DESCRIPTION OF THE FIGURES

FIG. 1 shows the results of Example 1 from our proprietary glucose metabolism assay. The mean ±Standard Error Mean (SEM) Statistical analysis was performed using One-way ANOVA (Tukey's multiple comparisons test). Different letter depict a statistically significant difference.

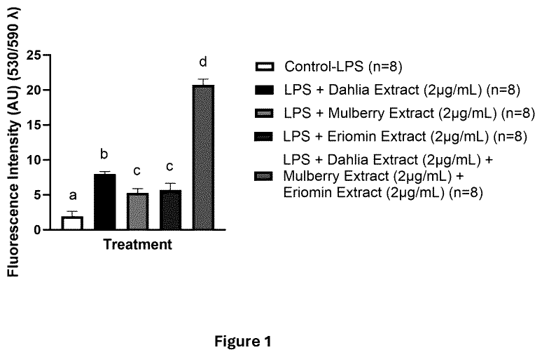

FIG. 2 shows the results of Example 2 from our proprietary glucose metabolism assay. The mean ±Standard Error Mean (SEM) Statistical analysis was performed using One-way ANOVA (Tukey's multiple comparisons test). Different letter depict a statistically significant difference.

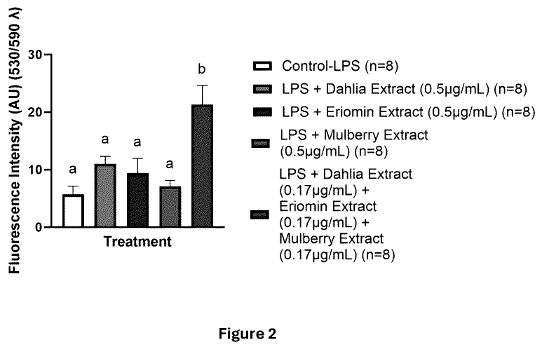

### DETAILED DESCRIPTION

All terms and definitions explained throughout the text of the specification relate to all examples and aspects disclosed herein, unless otherwise specified.

In one aspect there is provided a composition comprising:
a. dahlia extract
b. eriocitrin
c. mulberry extract.

In another aspect, there is provided a method of treating or preventing diabetes and/or one or more complications thereof comprising administering to a subject in need thereof a composition comprising:
a. dahlia extract
b. eriocitrin
c. mulberry extract.

In another aspect, there is provided the use of a composition comprising
a. dahlia extract
b. eriocitrin
c. mulberry extract
   in the manufacture of a medicament for the treatment or prevention of diabetes and/or one or more complications thereof.

In another aspect, there is provided a composition for use in the treatment or prevention of diabetes and/or one or more complications thereof comprising:
a. dahlia extract
b. eriocitrin
c. mulberry extract.

In one aspect the dahlia extract is a dahlia flower extract.

In one aspect the dahlia flower extract is Dahlia4®.

In one aspect the mulberry extract is a mulberry leaf extract.

In one aspect the eriocitrin is from Eriomin™.

As used herein, the terms “dahlia extract” and “dahlia flower extract” refer to a composition comprising various flavonoids prepared from yellow dahlia flowers (Dahlia pinnata). See for example Life Metabolism, Volume 2, Issue 4, August 2023.

In a preferred example, the dahlia extract is Dahlia4®.

Dahlia4® is an extract prepared from yellow dahlia flowers that contains a blend of yellow flavonoids. The predominant flavonoids are apigenin, butein, sulfuretin and isoliquiritigenin.

Apigenin (4′,5,7-trihydroxyflavone), is found in many plants, and is a natural product belonging to the flavone class that is the aglycone of several naturally occurring glycosides.

Butein is a chalcone of the chalconoids. It can be found in Toxicodendron vernicifluum (or formerly Rhus verniciflua), Dahlia, Butea (Butea monosperma) and Coreopsis. It has antioxidative, aldose reductase and advanced glycation end products inhibitory effects.

Sulfuretin is an aurones flavonoid found in the extract of Rhus verniciflua. The extract of Rhus verniciflua is known in the traditional medicine and scientific community to have anti-cancer properties.

Isoliquiritigenin is a chalcone found in the extracts of plants and has been shown to have anti-inflammatory, antimicrobial and antioxidant activities.

Eriocitrin is a flavonoid commonly found in lemons and other citrus fruit having the common name (2S)-3′,4′,5-Trihydroxy-7-[α-L-rhamnopyranosyl-(1→6)-β-D-glucopyranosyloxy]flavan-4-one.

In a preferred example, eriocitrin is from Eriomin™. Eriomin™ is a citrus lemon extract from lemon fruit or lemon peel or a combination thereof comprising around 70% eriocitrin.

Eriomin™ is a lemon extract that has been standardised to contain high levels of eriocitrin (eriodictyol 7-O-beta-rutinoside). It is composed of 70% eriocitrin, 5% hesperidin, 4% naringin and 1% didymin.

As used herein, the terms “mulberry extract” and “mulberry leaf extract” refer to a preparation prepared from the entire mulberry plant or from mulberry leaves. In one example mulberry extract and mulberry leaf extract refers to a water and/or ethanol solvent extracted product comprising 1-deoxynojirimycin.

The terms “diabetes mellitus” and “diabetes”, as used herein, refer to the metabolic disease classified in the International Classification of Diseases, Tenth Revision (ICD-10-CM), under the code E10-E14, and includes type 1, type 2, maturity-onset diabetes of the young (MODY), gestational diabetes, neonatal diabetes, steroid-induced diabetes, and pre-diabetes.

The term “therapy”, as used herein, refers to any treatment which is designed to cure, alleviate, remove or lessen the symptoms of, or prevent or reduce the possibility of malfunction of the subject body.

The term “treating” or “treatment” refers to the administration of a medicament, composition or compound to a patient to cure, alleviate, remove or lessen the symptoms of, or prevent or reduce the possibility of malfunction of the subject body.

The term “preventing” refers to preventing or reducing the possibility of developing diabetes mellitus and/or a complication thereof as herein defined.

The term “complications thereof” as used herein includes, but is not limited to diabetic retinopathy, diabetic nephropathy, diabetic skin ulceration, and diabetic neuropathy. Diabetic retinopathy is caused by damage to the blood vessels in the retina of the eye and can result in loss of visual acuity and eventual blindness.

The compositions described herein can be administered by different routes, e.g. orally, parenterally, by inhalation spray, buccally, sublingually, nasally, rectally, or parenterally, with oral route being preferred. The term “parenteral”, as used herein, includes subcutaneous, intracutaneous, intravenous, intramuscular, intrathecal, intralesional and intracranial injection or infusion techniques.

The term “an effective amount”, as used herein, refers to the amount of the active agent that is required to confer the intended therapeutic effect in the subject. Effective amounts may vary, as recognized by those skilled in the art, depending on route of administration, excipient usage, medical conditions, and the possibility of co-usage with other agents.

The effective amount of Eriomin™ may be from about 10 mg/day to about 3000 mg/day. The effective amount of mulberry leaf extract may be from about 10 mg/day to about 3000 mg/day. The effective amount of Dahlia4® may be about 10 mg/day to about 3000 mg/day.

The term “subject”, as used herein, refers to human and non-human mammals.

Effective amounts of the components of the compositions described herein can be administered to the same subject one time or multiple times.

Effective amounts of the compound of the compositions described herein can be administered to the same subject for one day or longer.

The compositions described herein can contain optional ingredients. Such optional ingredients generally are used individually at levels from about 0.01 to about 99.99 per cent by weight of the composition. Examples of suitable optional ingredients include, but are not limited to, excipients, carriers, minerals, carbohydrates, lipids, vitamins, co-factors, buffers, flavours and sweeteners, inorganic salts, cations and anions, taste modifying and/or masking agents, amino acids, organic acids, antioxidants, preservatives, colorants, and other suitable ingredients.

In some examples, the compositions described herein can contain microcrystalline cellulose, magnesium stearate, and colloidal silicon dioxide.

In some examples, the compositions can be formulated in different dosage forms. Nonexclusive examples of such forms include an orally acceptable dosage form, an injectable dosage form, an inhalation and an intranasal dosage form, and a rectal dosage form.

The oral composition can be any orally acceptable dosage form including, but not limited to, capsules, tablets, powders, aqueous solutions, suspensions, emulsions, and dispersions. Non-exclusive examples of carriers for tablets include lactose and corn starch. Non-exclusive examples of lubricating agents include magnesium stearate. Non-exclusive examples of diluents for oral administration in a capsule form include lactose and dried corn starch. When aqueous suspensions or emulsions are administered orally, the active ingredient can be suspended or dissolved in an oily phase combined with suspending or emulsifying agents.

In one example, the capsule is a vegetable capsule.

The sterile injectable composition can be any acceptable dosage form including, but not limited to, a sterile injectable solution or suspension in a parenterally acceptable diluent or solvent, for example, as a solution in pharmaceutical water, propylene glycol, mannitol, Ringer's solution, isotonic sodium chloride solution. In addition, sterile oils such as synthetic mono- or diglycerides, fatty acids, such as oleic acid and its glyceride derivatives, can be conventionally used as a solvent or suspending medium. Such oil solutions or suspensions can also contain a long-chain alcohol or carboxymethyl cellulose as a diluent or dispersant.

The inhalation and intranasal composition can be any acceptable dosage form including, but not limited to, solutions and dispersions in water or saline that may contain suitable preservatives, absorption promoters to enhance bioavailability, and/or other solubilising agents known in the art.

The rectal composition can be prepared according to procedures well known in the art in form of suppositories comprising the active ingredient and base capable to be melted or dissolved at a human body temperature to release the active ingredient. Non-exclusive examples of such bases include cacao butter, polyethylene glycol, and synthetic esters of glycerol like Witepsol bases.

The compositions described herein can be prepared by procedures well-known from the art. Such procedures include, but are not limited to, mixing the compositions described herein with other ingredients of the composition in conventional manner. Guidance for the preparation of compositions of the invention can be found in “Remington: The science and practice of pharmacy” 20th ed. Mack Publishing, Easton PA, 2000 ISBN 0-912734-04-3 and “Encyclopaedia of Pharmaceutical Technology”, edited by Swarbrick, J. & J. C. Boylan, Marcel Dekker, Inc., New York, 1988 ISBN 0-8247-2800-9 or a newer edition.

The following examples are illustrative only and are not intended to limit the scope of the disclosure in any way.

### EXAMPLES

The inventors assessed the individual and combined effects of dahlia extract, eriocitrin, and mulberry leaf extract on glucose metabolism in zebrafish. Glucose metabolism was assessed using a high-throughput metabolic assay that detects the ratio of NADH₂ to NAD+. This ratio acts as a readout for glucose metabolism as NADH₂ is produced when glucose is metabolised during glycolysis. Inflammation induced by liposaccharide (LPS) plays a key role in the development of type2 diabetes. LPS was used to impair blood sugar regulation. The signal produced by the glucose metabolism assay was quantified using a spectral photometer.

### Example 1

Zebrafish larvae were exposed to LPS, mulberry leaf extract, eriocitrin, and dahlia extract at concentrations of 2 μg/mL, followed by a glucose metabolism assay. We established that a dose of 2 μg/mL was the threshold dose for dahlia extract to improve glucose metabolism. This was required because extract at a higher dose of 10 μg/mL maximally activated glucose metabolism and a ceiling effect was reached that could not be exceeded by addition of eriocitrin or mulberry leaf extract.

Experimental groups were as follows: LPS, LPS and Dahlia4® (2 μg/ml), LPS and Eriomin™ (2 μg/mL), LPS and Mulberry leaf extract (2 μg/ml), and LPS, Dahlia4®+Eriomin™+Mulberry leaf extract (2 μg/ml each).

Quantitative assessment showed that all three nutraceuticals despite the relatively low dose increased glucose metabolism in isolation with Dahlia4® being significantly more effective than Eriomin™ and mulberry leaf extract. Treatment with LPS and Dahlia extract increased glucose metabolism by 309% (P value <0.0001), LPS and Mulberry extract by 172%, (P value 0.0174), LPS and Eriomin™ extract by 193% (P value 0.0059) relative to the baseline control group. Treating zebrafish with LPS, Dahlia 4®, Eriomin™ and Mulberry Leaf extracts led to a 963% increase in glucose metabolism compared to the baseline control group (P value <0.0001). This confirms that a combination of the three components showed a marked elevation in glucose metabolism suggesting a synergistic effect.

### Example 2

Example 1 used doses of the individual components of the composition as described herein at the threshold of effectiveness. All were still effective at the dose used and improved glucose metabolism in isolation. Example 2 was designed to test synergism by combining all three nutraceuticals at a subthreshold dose. The experimental design was identical to that from Example 1 except that the following subthreshold doses were used: LPS, LPS and dahlia extract (0.5 μg/mL), LPS and eriocitrin (0.5 μg/mL), LPS and mulberry extract (0.5 μg/mL), and LPS, dahlia extract (0.17 μg/mL), eriocitrin (0.17 μg/mL) and mulberry extract (0.17 g/mL). In this example the combination dose of all three components was not higher than the dose tested in isolation.

At 0.5 μg/mL none of the components significantly increased glucose metabolism in isolation. However, the combination of the Dahlia4®, Eriomin™ and Mulberry leaf extracts at 0.5 μg/mL led to a 274% increase in cell metabolism compared to the baseline (P value <0.0001), clearly confirming a synergistic effect to boost glucose metabolism of all three nutraceuticals.

### CONCLUSION

We have demonstrated that a combination of dahlia extract, mulberry extract and eriocitrin synergistically increases glucose metabolism. We hypothesise This profound effect is likely caused by the fundamentally different mode of action of the three nutraceuticals targeting type 2 diabetes at the level of glucose uptake (mulberry leaf extract), insulin secretion (Eriomin™) and long-term blood sugar regulation (Dahlia4®).

1. A composition comprising:
a. dahlia extract
b. eriocitrin
c. mulberry extract.

2. The composition of claim 1, wherein the dahlia extract is a dahlia flower extract.

3. The composition of claim 1, wherein the mulberry extract is a mulberry leaf extract.

4. The composition of claim 1, wherein the eriocitrin is from Eriomin™.

5. A method of treating or preventing diabetes and/or one or more complications thereof comprising administering to a subject in need thereof a composition comprising:
a. dahlia extract
b. eriocitrin
c. mulberry extract.

6. The method of claim 5, wherein the dahlia extract is a dahlia flower extract.

7. The method of claim 5, wherein the mulberry extract is a mulberry leaf extract.

8. The method of claim 5, wherein the eriocitrin is from Eriomin™.

9. A method of preparing a composition for treating diabetes and/or one or more complications thereof, comprising: combining dahlia extract, eriocitrin, and mulberry extract.

10. The method of claim 9, wherein the dahlia extract is a dahlia flower extract.

11. The method of claim 9, wherein the mulberry extract is a mulberry leaf extract.

12. The method of claim 9, wherein the eriocitrin is from Eriomin™.

13-16. (canceled)

17. The composition of claim 2, wherein the mulberry extract is a mulberry leaf extract.

18. The composition of claim 2, wherein the eriocitrin is from Eriomin™.

19. The composition of claim 3, wherein the eriocitrin is from Eriomin™.

20. The method of claim 6, wherein the mulberry extract is a mulberry leaf extract.

21. The method of claim 6, wherein the eriocitrin is from Eriomin™.

22. The method of claim 7, wherein the eriocitrin is from Eriomin™.
